(19) United States
(12) Patent Application Publication
(10) Pub. No.: US 2010/0168561 A1
(43) Pub. Date: Jul. 1, 2010

(54) Intraoperative Tissue Mapping and Dissection Systems, Devices, Methods, and Kits

(75) Inventor: Rolfe C. Anderson, Saratoga, CA (US)

Correspondence Address: WILSON, SONSINI, GOODRICH & ROSATI, 650 PAGE MILL ROAD, PALO ALTO, CA, 94304-1050, US

(73) Assignee: TRILLIUM PRECISION SURGICAL, INC., Saratoga, CA (US)

(21) Appl. No.: 12/519,495

(22) PCT Filed: Dec. 18, 2007
(86) PCT No.: PCT/US2007/087962

§ 371 Date: Nov. 30, 2009

Related U.S. Application Data
(60) Provisional application No. 60/875,355, filed on Dec. 18, 2006.
(60) Provisional application No. 60/992,985, filed on Dec. 6, 2007.

(51) Int. Cl. A61B 5/05

(52) U.S. Cl. 600/424

(57) ABSTRACT

Intraoperative devices are described that assist the surgeon in identifying the location and characteristics of tissues and structures. Devices are also described that have the added capability of marking the location of the identified tissues and structures. This invention also includes devices that can selectively ablate adjacent tissues while avoiding damage and trauma to the identified tissues and structures by combining ablation with sensing, where sensing of either tissue properties, markings made by another device or surgeon, or a reference probe can be used. Devices are also described that protect tissue in the proximity of reference markings or probes by closed loop inhibition of the ablation process. The devices, systems, methods and kits described are adapted and configured to facilitate locating a target structure or target tissue within a body of a mammal, including nerves, peripheral nerves, blood vessels, and tubes such as the ureter. The devices, systems and methods may discriminate between different tissues by exploiting the electrical, mechanical, and physiological properties of the body.

### CROSS-REFERENCE

This application claims the benefit of U.S. Provisional Application No. 60/875,355, filed Dec. 18, 2006, by Rolfe Carter Anderson entitled Surgical Assistance Systems, and U.S. Provisional Application 60/992,985, filed Dec. 6, 2007 by Rolfe C. Anderson entitled Closed Loop Dissection Using Reference Probes, which applications are incorporated herein by reference.

### BACKGROUND OF THE INVENTION

This invention relates generally to surgical navigation and control. In particular, the invention provides intraoperative devices that assist the surgeon in identifying the location and characteristics of tissues and structures. Devices are also described that have the added capability of marking the location of the identified tissues and structures. This invention also includes devices that can selectively ablate tissues that are adjacent or surrounding a target identified tissue while avoiding damage and trauma to the identified tissues and structures by combining ablation with sensing, where sensing of either tissue properties, markings made by another device or surgeon, or a reference probe can be used. Devices are also described that protect tissue in the proximity of reference markings or probes by closed loop inhibition of the ablation process.

Proper identification of anatomical features during surgery is critical for achieving positive outcomes and avoiding complications. Veins, arteries and nerves can be difficult to distinguish from one another in situ. Blunt dissection is often used to carefully peel apart natural separation planes between different tissue, to minimize bleeding and trauma and improve anatomical navigation. This approach fails when proper dissection planes are not identified. Better identification of natural dissection planes would be valuable for this purpose. Searching for the location of specific nerves and vessels can consume significant surgical time and carries the risk of injury to delicate structures. Current practice relies upon anatomical landmarks, but anatomical variations, disease, trauma and scar tissue can slow the process of identifying anatomical features and increase the risk of injury. Unintended damage to these structures can result in significant complications. Trauma to sensor nerves can result in numbness and loss of function. Damage to motor nerves can result in loss of function.

Nerve identification and mapping is important in many surgeries such as thyroid and parotidectomy. Motor nerve monitoring has been identified as critical for procedures such as skull base tumor removal anterior, microvascular decompression for trigeminal neuralgia, large posterial fossa tumor removal, acoustic neuroma removal, facial nerve decompression, facial trauma repair, mastoidectomy, congenital atresia, cochlear implantation, carotid body tumor removal, carotid endarterectomy, radical neck dissection, and thyroidectomy.

For example, in parotidectomy surgery the facial-nerve injury is common, with 17% to 100% of patients experiencing transient paralysis of all or part of the facial nerve. Identification of the facial nerve matrix is critically important and the nerve branching within the parotid can be quite complex. A retrograde approach may be desirable or required when the main nerve trunk cannot be exposed, so that the surgeon works first distally, finding a peripheral nerve branch and then dissecting proximally. However, with this approach the risk of nerve trauma is elevated further because unambiguous identification of nerve segments is difficult.

Other examples of surgeries that have significant risk of collateral damage, include, for example, pelvic surgery, spine surgery, and radical prostatectomy which carries a significant risk of impotence and the potential for incontinence due to trauma to the nerves adjacent or surrounding the prostate and trauma to the urethral sphincters. Locating the ureter during pelvic surgery can be time consuming and carries the risk of injury, particularly when there is scarring or tumors in the adjacent or surrounding tissue. As will be appreciated by those skilled in the art, injury to the ureter during pelvic surgery can result in impaired function, infection, and other complications. Also compelling is the importance of identifying nerves during back or spine surgery. Better discrimination between diseased and normal tissue, with cancer surgery for example, might be used to more completely dissect tumor tissue and achieve better tumor margins while minimizing excess tissue removal and the risk of complications.

Some nerves can be particularly sensitive to surgical trauma. For example, nerve branches to the eye should be dissected with particular care: even transient weakness of these branches may have a significant impact on morbidity. When dissecting certain structures such as nerves it can be important to preserve the associated vasculature that supports them, particularly with the neurovascular bundles of the prostate.

Surgical procedures can result in potentially avoidable complications. For example, the radical retropubic prostatectomy procedure includes dissection and anastamosis of the urethra. Incontinence, urethral strictures, and longer recovery times are a direct result of this practice. Significant complication rates related to the urethra in this procedure include anastomosis leakage, urinary retention, and anastomosis structure (10%, 4.6% and 2.5%, respectively).

When tumors are in close proximity to important structures such as critical nerves, the surgeon might damage these structures to ensure tumor removal. Generally, it is appreciated that improvements in the identification and selective dissection of tissues and structures can improve surgery and reduce complications. For example, intraoperative neural probes have been introduced into the marketplace that a surgeon can use to locate and identify specific nerves. Examples include the NIM PRS and NIM spine from Medtronic (Minneapolis Minn.) and the Orthomon from Axon Systems (Hauppauge N.Y.).

Intraoperative ultrasound has also been employed to identify structures, but does not discern between specified vessels and hasn't been employed for closed loop control of ablation.

### SUMMARY OF THE INVENTION

An aspect of the invention is directed to an intraoperative device for detecting a spacing between a plurality of tools used on a target area of tissue during surgery. The device comprises a probe element in communication with a signal generator; and a reference element in communication with a signal generator wherein the reference element is positionable within a detectable signal range. One or more reference elements can be used, as desired. Spacing between the probe element and the reference element can be determined by, for example, measuring a characteristic of a detectable signal. Additionally, a dissection element can be provided that is modulatable in response to a spacing between the probe element and the reference element. Dissection elements can be any suitable element adapted and configured to be modulatable in response to spacing, including, for example, one or more of an ultrasonic source, an electroablation probe, vibrating blade, cryoablation probe, thermal ablation probe, a plasma source, and a laser. A marking element can also be provided that is adapted and configured to identify and mark a location of a target tissue. In some configurations, a notification element may further be provided for providing a sensory notification of a proximity between the reference electrode and a second structure such as the target area of tissue or any structure identified by the surgeon as being of interest. The second structure can also be, for example, a marking located on or adjacent to the target area of tissue, and/or a probe element. Various detectable signals can also be generated by the probe element, the reference element, or both the probe and reference elements. Signals include, for example, magnetic signals, optical signals, acoustic signals, thermal signals, and/or any other suitable detectable signal. These detectable signals can be detected by the probe element, the reference element, both the probe and reference element, along with, or in lieu of, any component of the system adapted and configured to detect signals. In some configurations, the reference element is configured to have detectable properties, such as magnetic properties, electrical properties, radioactive properties, optical properties, acoustic properties, and/or thermal properties. The reference element can also be operably connected to a suitable power source such as an electromagnetic radiation power source. The power source may, in some cases, be external to the device. Additionally, the reference element can be operably connected to a signal generator adapted and configured to generate a signal, such as magnetic signals, optical signals, acoustic signals, thermal signals, and/or any other suitable signals. In some configurations, the device can be adapted and configured for use in, for example, laparoscopic or minimally invasive surgery and by comprising a catheter for deploying the reference element.

A method is provided for detecting a spacing between a probe element and a target tissue. The method comprises the steps of placing a reference element within a detectable signal range of the target tissue; generating a detectable signal; detecting a signal; and determining a spacing between the probe element and the reference element from a characteristic of the detected signal. The method can further include the step of dissecting tissue, e.g. tissue adjacent or surrounding the target tissue, using a dissection element. For example, the dissection element can be modulated in response to a spacing between the probe element and the reference element. Additionally, a user can be notified of the spacing between the probe element and the reference element, for example, by using sensory notification, such as would be achieved by visual, audible, or tactile output. Furthermore, a marking element can be activated to mark the location of the reference element and target tissue, such as a urethra, or other identified target tissue. Also, the reference element can be placed within the urethra using, for example, a catheter. As will be appreciated by those skilled in the art, the detectable signal can be generated by the probe element, the reference element, or both the probe and reference element. Additionally, the probe element, the reference element, or the probe and reference element, can be configured to sense the detectable signal. In some aspects, the reference element can be configured to have detectable properties. Additionally, the step of placing the reference element can occur prior to generating a detectable signal including, for example, during a different procedure. In some instances it may be desirable to use intraoperative imaging techniques to position the reference element during these methods.

Another aspect of the invention is directed to a system for detecting the distance between a probe and reference electrode. The system comprises a probe element configured for communication with a signal generator; a reference element configured for communication with a signal generator wherein the reference element is positionable within a detectable signal range; and a signal generator for generating a detectable signal that is in communication with the probe element and the reference element.

Still another aspect is directed to a kit for detecting the distance between at least two tissue structures at a surgical site. The kit includes, for example, a probe element with a signal generator; and a reference element in communication with a signal generator wherein the reference element is positionable within a detectable signal range. The kit can also include a power supply, a set of instructions and any other components that would be useful to the end user.

An intraoperative device for marking tissue during a surgical procedure is also provided. The device comprises a sensing element for detecting a detectable signal; and a marking element for creating a detectable mark associated with a location on the tissue. Furthermore, the marking element can be adapted and configured to comprise a pump dispenser and a dispensing aperture. In that configuration, the dispensing aperture could be configured to be fluidly connected to the pump dispenser. Depending upon the application of the device, the sensing element could be a nerve monitoring probe. Furthermore, the dispensing aperture can be associated with the sensing element. In some cases, using a dye as a detectable mark is desirable, for example, methylene blue, India ink, India ink in isotonic saline solution, or any other suitable dye. Alternatively, or in conjunction with the dye, the detectable mark can be a wax, such as a paraffin wax. Where wax is used, the marking element would be configured for heating in order to facilitate use of the wax as a marker. Furthermore, in some aspects, the location of the detectable mark might be stored in a computer, projected on a digital display, or used with a computerized surgical system. Additionally, the detectable marks can be marks that are capable of being projected onto the surgical site.

A system for marking tissue during a surgical procedure is also provided. The system comprises a nerve monitoring probe for detecting a signal generated by a nerve; a marking element for creating a detectable mark associated with the location of the nerve to mark the location of the nerve; and a controller unit for activating the marking element in response to a signal from the nerve monitoring probe.

Still another aspect of the invention is directed to a method for creating a marking associated with the location of a tissue of interest at a surgical site. The method comprises probing a tissue with a sensing element; detecting a signal generated by the tissue being probed; characterizing the tissue being probed to determine if the tissue is a tissue of interest; activating the marking element to mark the tissue if the tissue is of interest; and marking the tissue with a marking element. The tissue of interest can be any of tissue of interest including, for example, a nerve, a nerve bundle, a vein, an artery, a ureter, a muscle, a urethra, or any other suitable fascicle, tube, lymphatic vessel, blood vessel, or any other suitable tissue.

Another kit contemplated is for marking tissue during a surgical procedure. The kit comprises a dispensing aperture for affixing to a nerve monitoring probe; a biocompatible marking substance; and a marking element for creating a detectable mark associated with a location on the tissue. The kit can further comprise any additional components that would make the kit useful to an end user including, for example, a dispenser in fluid communication with the marking element, and/or a set of instructions.

Still another device is an intraoperative device for mapping an area of tissue during a surgical procedure. This device comprises at least one excitation element for interacting with a tissue of interest with a stimulus to generate a detectable signal; at least one sensing element for detecting a presence or absence of a signal; and a marking element for creating a detectable mark associated with a location on a tissue. The tissue of interest can be characterizable by, for example, using electrical stimulation and electrical detection. Furthermore, the detectable signal may also be capable of characterizing the tissue. Additionally, the method can include the step of determining whether the tissue is a tissue of interest prior to marking the tissue. In some cases, the tissue of interest may be characterizable by a measure of electrical impedance, for example, between the at least one excitation element and the at least one sensing element. Thus, for example, the measure of electrical impedance can be used as an indicator of whether a tissue of interest is located between said at least one excitation element and at least one sensing element. Furthermore, the detectable signal is an electrical signal. The excitation electrode can be used to stimulate the tissue of interest with one or more of an alternating voltage stimulus, with a depolarizing voltage stimulus or with a non-depolarizing voltage stimulus. Furthermore, the excitation element can stimulate the tissue of interest with a voltage between about 10 Hz and about 1 MHz, or between about 10 Hz and about 10 kHz. A plurality of excitation elements and a plurality of sensing elements can be used to form a plurality of electrode pairs. In that case, a measure of impedance between the plurality of electrode pairs is sequentially measured.

An intraoperative device for mapping an area of tissue during a surgical procedure is also provided. This device comprises at least one excitation element for interacting with a tissue of interest with a stimulus to generate a detectable signal; a remote interrogation element; and a marking element for creating a detectable mark associated with a location on a tissue. The detectable signal can be, for example, a measure of the electrical impedance measured between the excitation element and the remote interrogation element. Additionally, the excitation element can be moveable. Furthermore, in some aspects, the detectable signal can be a measure of the electrical impedance measured between the remote interrogation element and a plurality of moveable excitation electrodes. The marking element or elements can be adapted and configured to create the detectable mark associated with the location of a tissue of interest. Furthermore, the excitation element stimulus for stimulating the tissue of interest can be any of a variety of suitable stimulus, including, for example, electrical stimulation, magnetic stimulation, mechanical stimulation, acoustic stimulation, optical stimulation, thermal stimulation, electromagnetic stimulation, mechanical vibration, ultrasound, stimulus arrays, and imaging. The detectable signal detected can be an electrical signal, a mechanical signal, an electromagnetic signal, a magnetic signal, a thermal signal, ultrasound, detection arrays, imaging methods, or any other suitable detectable signal. Additionally, the detectable mark could, in some cases, be a surface cautery of the tissue of interest, and/or a dye selected from India ink, Prussian blue, crystal violet, or any other suitable dye. In some instances, fluorescent dye or radioactive material may be desirable for use as a detectable mark. In other cases, the detectable mark might be a particle, a quantum dot, a carbon nanotube, a paramagnetic particle, a ferromagnetic particle, a metallic particle, a radioactive particle, or a colored particle. Paraffin wax, wax, sucrose solution, or any other suitable material that forms a gel upon deposition are also suitable for use as a detectable mark. Furthermore, optical marks, or marks made by an electrochemical reaction can be used without departing from the scope of the invention. The marking element can be further adapted and configured to create multiple detectable marks on the tissue of interest. Each of the multiple detectable marks can further identify multiple different types of tissues. Furthermore, different types of marking elements can be used to mark each of the discrete tissue types such that the sensory signal enables the user to distinguish between the different tissue types.

An intraoperative device for mapping an area of tissue during a surgical procedure is provided comprising: an excitation element for stimulating an area of tissue with a stimulus to create a detectable signal; a sensing element for detecting the detectable signal; and a notification element for providing a sensory signal to a user in response to detecting a spacing between the sensing element and the area of tissue. The sensory signal can be a visual signal, an auditory signal, or a tactile signal, or a combination thereof.

A method for creating a mark associated with a location on a tissue of interest is included which comprises the steps of stimulating a tissue of interest with a stimulus generated by an excitation element thereby creating a detectable signal; sensing the detectable signal with a sensing element; characterizing the tissue of interest by analyzing the detectable signal; and activating a marking element to mark the tissue of interest if desired, wherein the marking element creates a mark associated with the tissue identification and location. The tissue of interest can be any tissue identified by the user, such as one or more of a nerve, nerve bundle, vein, artery, ureter, muscle, urethra, or other fascicle, tube, lymphatic vessel, blood vessel.

A system for mapping an area of tissue during a surgical procedure is also provided comprising: at least one excitation element for interacting with a tissue of interest with a stimulus to generate a detectable signal; at least one sensing element for detecting a presence or absence of a signal; a marking element for creating a detectable mark on the tissue; and a dispenser for dispensing a marking material to the marking element.

Still another system is provided for mapping an area of tissue during a surgical procedure that comprises the steps of at least one excitation element for stimulating a tissue of interest with a stimulus to generate a detectable signal; a remote interrogation element; and a marking element for creating a detectable mark associated with a location on a tissue.

Additional kits include a kit for mapping an area of tissue during a surgical procedure comprising: at least one excitation element for interacting with a tissue of interest with a stimulus to generate a detectable signal; at least one sensing element for detecting the presence or absence of a signal; a marking element for creating a detectable mark on the tissue; and a dispenser for dispensing a marking material to the marking element. Other components can be added to the kit including, for example, a set of instructions for the user or operator.

An additional kit is directed to a kit for mapping an area of tissue during a surgical procedure comprising: at least one excitation element for stimulating a tissue of interest with a stimulus to generate a detectable signal; a remote interrogation element; and a marking element for creating a detectable mark on a tissue. Instructions, and other useful components, can also be provided.

An intraoperative device for selective dissection of tissue is also provided comprising: a detectable mark associated with a location on a tissue of interest, thereby identifying and marking the location of the tissue; and a dissection element for selectively dissecting an area of tissue adjacent the marked tissue. The dissection element can further comprise a closed loop dissection element for protecting the tissue surrounding an area of tissue to be dissected. Furthermore, the detectable mark can be selected to affect the trajectory of the dissection element, or the action of the dissection element. Detectable marks can include, for example, wax, a dielectric material, a foam, or any other suitable material. The dissection element can also be adapted to dissect an area of tissue surrounding the marked tissue through ablation. In an additional aspect, the device additionally comprises a detection element. The detection element can be adapted to detect optical marks, electrical markings, radioactive, or magnetic markings on the marked tissue.

A device for selective dissection of tissue is provided comprising: a tissue characterization system for identifying a tissue of interest; and a dissection element for dissecting a tissue surrounding the tissue of interest. The tissue characterization system can further comprise an excitation element and a sensing element.

In still another aspect, an intraoperative device is provided for selective dissection of tissue comprising: a detectable mark associated with a location on a tissue of interest, thereby identifying and marking the location of the tissue; and a dissection element for selectively dissecting the marked tissue.

A method for selectively removing tissue at a surgical site comprises another aspect of the invention. This method comprises: marking a tissue of interest with a detectable mark using a marking element; detecting the mark using an integrated probe-dissection element wherein the probe component detects the detectable mark; dissecting the marked tissue using the integrated probe-dissection element wherein the dissection component dissects the marked tissue.

Still another method is provided for selectively removing tissue at a surgical site. This method comprises: positioning a reference element within or adjacent to a tissue of interest; probing the area surrounding the tissue of interest using a probe element wherein a dissection element is operably connected to the probe element; detecting the location of the reference element with the probe element; and dissecting the tissue the reference element thereby protecting the tissue nearest the reference element.

Yet another aspect is directed to a method for selectively removing tissue at a surgical site comprising: marking a tissue of interest with a detectable mark using a marking element; detecting the mark using an integrated probe-dissection element wherein the probe component detects the detectable mark; dissecting the tissue adjacent the mark using the integrated probe-dissection element wherein the dissection component dissects the tissue.

Another method for selectively removing tissue at a surgical site comprises: positioning a reference element within or adjacent to a tissue of interest; probing the area adjacent the tissue of interest with a probe element wherein the probe element stimulates the tissue to generate a detectable signal; sensing the detectable signal using the reference element; detecting a spacing between the probe element and the reference element by analyzing a characteristic of the detectable signal; and dissecting the tissue adjacent the reference element with a dissection element wherein the dissection element is capable of being modulated by the spacing between the probe element and the reference element.

Still another aspect of the invention is directed to a system for selectively dissecting tissue comprising: a marking element for creating a detectable mark associated with a location of a tissue of interest, thereby identifying and marking the location of the tissue; and a dissection element for selectively dissecting an area of tissue adjacent the marked tissue. Additional elements can be provided in the system including, for example, a mapping element, a proximity system, and a notification element. Additionally, the system can further include ultrasound capability, an array, or other imaging system functionality, as might be desirable or appropriate under the circumstances.

Additionally, an aspect of the invention is directed to a kit for the selective dissection of tissue comprising: a marking element for creating a detectable mark on a tissue of interest, thereby identifying and marking the location of the tissue; and a dissection element for detecting a marking and selectively dissecting an area of tissue adjacent the marked tissue. The kits can comprise additional components that are useful to the end use or operatory. For example, the kit can also comprise, a mapping element, a proximity system, and/or a notification element. Additionally, the kit can include an ultrasound device, an array, and/or imaging systems.

Yet another aspect of the invention is directed to a kit for selective dissection of an area of tissue during a surgical procedure. The kit comprises, for example, at least one excitation element for stimulating a tissue of interest with a stimulus to generate a detectable signal; at least one interrogation element; and a controllable dissection element.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the surgical site of the prostate and adjacent anatomical structures.

FIG. 2 illustrates a surgical procedure where the bladder neck is dissected near the prostate.

FIG. 3 illustrates a surgical plane along which the neurovascular bundle innervating the prostate will be dissected from the prostate.

FIG. 4A illustrates a closed loop marking probe of an intraoperative mapping system; FIG. 4B illustrates a selective dissection probe of an intraoperative dissection system.

FIG. 5 illustrates a proximity dissection probe dissecting tissue outside of the proximity limit.

FIG. 6 illustrates an intraoperative marking device system.

FIG. 7 illustrates a “sweep-type” mapping system for intraoperative detection and marking of nerves and other structures.

FIG. 8 illustrates a mapping array for use with an intraoperative mapping device.

FIG. 9A illustrates a plan view of an alternative mapping array for use with an intraoperative mapping system; FIG. 9B illustrates a cross-sectional view of the mapping array shown in FIG. 9A.

FIG. 10A illustrates a brush-type marking system for intraoperative detection and marking of tissues; FIG. 10B illustrates a cross-sectional view of the mapping element shown in FIG. 10A.

FIG. 11 illustrates a selective dissection system.

FIG. 12 illustrates a plan view array for use with a selective dissection system.

FIG. 13A illustrates a plan view of an alternate dissection array for use with a selective dissection system;

FIG. 13B illustrates a cross-sectional view of the dissection array shown in FIG. 13B.

FIG. 14 illustrates a proximity system in use on a target tissue.

FIG. 15 illustrates a top view of a dissection experiment.

### DETAILED DESCRIPTION OF THE INVENTION

The devices, systems, methods and kits described are adapted and configured to facilitate locating a target structure or target tissue within a body of a mammal, including nerves, peripheral nerves, blood vessels, lymphatic vessels and nodes, as well as tubes such as the ureter and urethra. The devices, systems, methods and kits facilitate detection of target tissue that is lying near, close, contiguous, adjoining or neighboring a tissue on which a procedure is to be performed; particularly tissue that would be of interest during a procedure because its proximity to the tissue on which the procedure is to be performed. The devices, systems and methods may discriminate between different tissues by exploiting electrical, mechanical, and physiological properties. As will be appreciated, the target structures include, for example, an elongate organ or tissue structure located within a tissue-under-test (TUT) or tissue on which a procedure, such as a surgical procedure, is being performed. Such target structures include, for example, nerves, nerve bundles, veins, arteries, ureters, muscles, urethras, or other fascicles, tubes, tubules, lymphatic or blood vessels. The TUT includes a portion of a mammalian body, or surgical site on which a mapping system or dissection system is being used. For example, in thyroid surgery the TUT is the thyroid and adjacent tissue. It is anticipated that the inventions described here may be used multiple times during the course of a surgical procedure and progressive dissection, so that the TUT may change.

By providing closed-loop dissection this invention allows surgeons to more aggressively and confidently excise all non-protected tissue, while improving the likelihood of both complete removal of the desired tissue (e.g., tumor) along with the preservation of functional tissue.

### I. DEVICE OVERVIEW

This invention encompasses the application of currently known tissue-characterization methods and includes, for example, intraoperative ultrasound, electromyography, and nerve-conduction testing. These methods can be used in conjunction with the systems and devices described herein. Additionally, thermal imaging, electromagnetic field sensing, nerve depolarization, nerve stimulation and electrical impedance mapping, can be used. For example, U.S. Pat. No. 6,609,018 entitled “Electrode array and sensor attachment system for noninvasive nerve location and imaging device” and U.S. Pat. No. 6,564,079 entitled “Electrode array and skin attachment system for noninvasive nerve location and imaging device” both describe electrical methods for identifying the locations of nerves through the surface of the skin.

It is anticipated that the shaping of an excitation signal could be used to improve detectability. For example, modulation of a signal at specific carrier frequencies, duty cycles, or other signal coding techniques can be used in any of the devices or systems. Also signal-processing techniques such as frequency filtering, autocorrelation, wavelet analysis, and neural networks may potentially be used to improve the signal to noise performance. Signals can include, for example, a physical propagation of an electrical current through the TUT. Furthermore, signals can encompass the result from either a stimulus or excitation provided by, for example, an excitation element or can be a signal intrinsic to the body and may be, e.g., a nerve depolarization pulse or the transfer of electrical current through a nerve, such as action potential propagation. Other signals that may be used in this invention include, but are not limited to, electrical current, nerve depolarization pulse, thermal conduction, physical vibration, acoustic wave, mechanical deformations or stresses, electromagnetic or optical transmission.

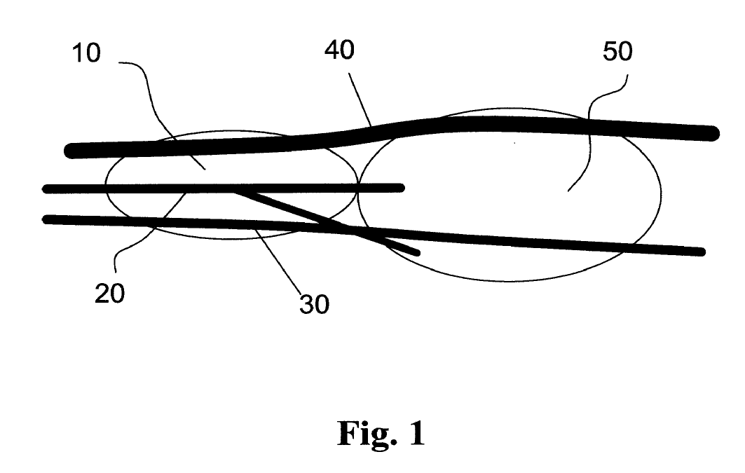

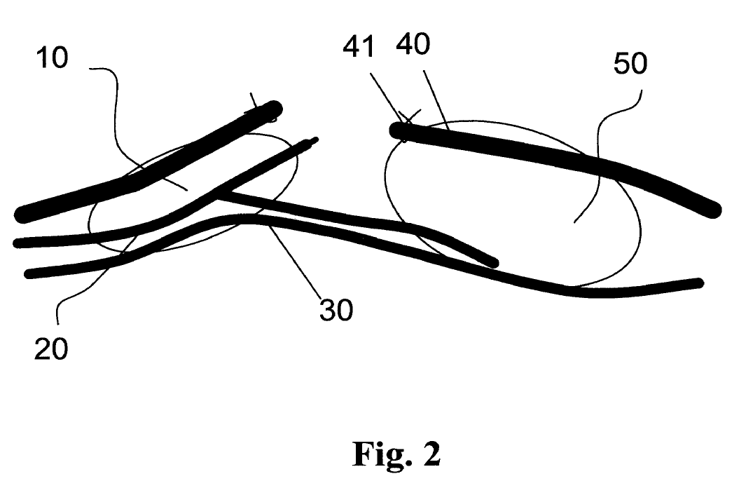

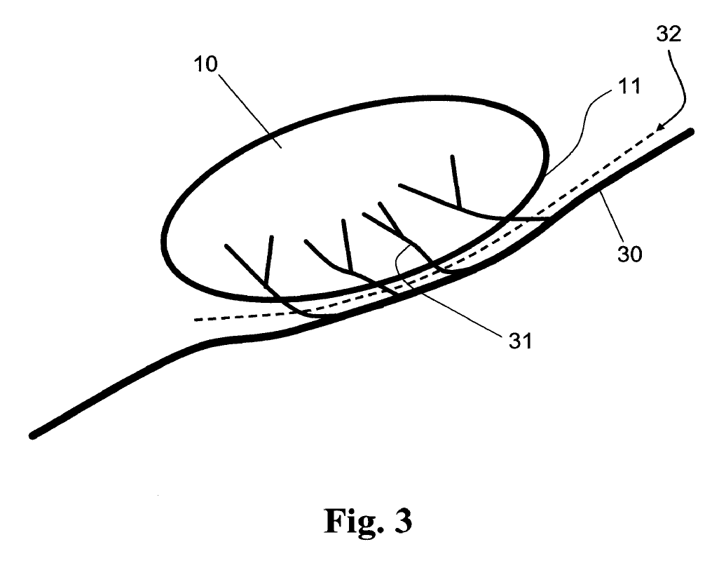

FIG. 1 illustrates an anatomical structure, like the prostate 10, surrounded by other anatomical features that might be damaged during a procedure, such as a radical prostatectomy, because of the proximity to other structures. Procedures include, for example, surgical procedures, minimally invasive procedures, endoscopic procedures, laparoscopic procedures, etc. In the area of the prostate 10, the urethra 20, the neurovascular bundles 30, dorsal vein 40, and bladder 50 can all hamper the ability to surgically access a target surgical site, such as a tumor, during a procedure. FIG. 2 illustrates the pelvic area after a bladder neck dissection near the dorsal vein 40. While the dorsal vein 40 has been cut and sealed 41 during this procedure, the urethra 20, neurovascular bundles 30, and bladder 50 all are in danger of being traumatized or damaged. FIG. 3 illustrates a surgical cut made during a prostatectomy procedure. In this example, a surgical incision 32 is made between the neurovascular bundles 30 and the branches of the neurovascular bundle 31 that innervate the prostate 10 at the capsule 11 of the prostate 10. Any damage or trauma to the neurovascular bundles 30 can result in impotence and other adverse complications. Again, as will be appreciated, the close proximity of the anatomical structures during these procedures increases the chance that undesirable trauma or damage may occur to one of the neighboring structures during a surgical procedure.

The intraoperative mapping systems described enable marking tissue during a surgical procedure. Marking can include, for example, any type of indicator on, adjacent, near, or communicable with a target structure. A marking identifies and indicates the location of one or more target structures within the TUT area. The intraoperative device usually comprises at least one excitation element for creating a detectable signal or interacting with a tissue of interest with a stimulus to generate a detectable signal. The signal can be, for example, a physical propagation of an electrical current through the TUT. Other signals include, but are not limited to, electrical current, nerve depolarization pulse, thermal conduction, physical vibration, acoustic, electromagnetic or optical transmission. The excitation element can be adapted and configured to, for example, create a signal that is detected by a sensing element for mapping a location of target tissues or vessels. The sensing element can include, for example, a device that is adapted and configured to detect a signal. Signal detection devices include, for example, voltage sensing, current sensing, magnetic field sensing, temperature sensing, vibration sensing, movement sensing, pressure sensing, electromagnetic radiation sensing, or optical sensing. Frequency filtering and other signal processing techniques may also be used to improve the signal to noise performance, as will be appreciated. Furthermore, sensing element may be made using conventional fabrication techniques such as molding or machining, or by micro-fabrication or micro electro-mechanical systems (MEMS). The sensing element can be placed on the probe, at any suitable location within the system, or at some other location of the body, if desired.

Additionally, the signal from the excitation element may be modulated in ways to improve detectability, for example, by modulating at specific carrier frequencies, duty cycles, or other signal coding techniques. The excitation element may also employ one or more electrodes, either monopolar or bipolar, magnetic solenoids, light emitting diodes (LEDs), thermal elements, acoustic energy, or localized mechanical deformations used to create a signal. Excitation elements may also be made using conventional fabrication techniques such as molding or machining, or by micro-fabrication or MEMS. The excitation element may be positioned on a probe, or placed in contact with another part of the body. Typically, the device comprises a sensing element for detecting the presence or absence of the signal and either a marking element for creating a detectable mark associated with a location on the tissue, or a dissection element for cutting or ablating the tissue

The marking element can, as will be appreciated, be any device that creates a mark. For example, marking elements can include, but is not limited to, ink-deposition systems, ink-jet arrays, electrode arrays for cautery marking or localizing electrodeposition. The marking element may be made using conventional fabrication techniques such as molding or machining, or by micro-fabrication or MEMS.

### II. MARKING SYSTEM

The intraoperative marking system described herein can be used to identify and mark a location of target structures located in the TUT surgical site. Furthermore, the marking system can be adapted and configured to work in conjunction with existing commercially available anatomic identification probes such as nerve-identification or ultrasound probes to create markings on target structures, e.g., using commercially available nerve-identification probes to detect and create markings on nerves. Typically, a marking system comprises a bolus-dispenser (similar to those used for dispensing adhesives for packaging) which is configured to be triggered by a signal from the nerve-identification probe system. A suspension of a marking material is loaded into, and dispensed from, the bolus dispenser at identified target structures. For example, the marking system can be used to create an ink pattern at the surgical site that indicates the locations of an identified target structure, such as the nerves near and/or through a surgical site.

The marking system can also be integrated with currently known methods for the controlling and/or patterning the deposition of marking materials on surfaces. Such methods include, but are not limited to, ink jet printing and metered dispensing. Also provided herein are marking systems that apply less conventional methods for controlling and/or patterning the deposition of materials on surfaces. Such methods include but are not limited to patterned surface cautery and electrochemical deposition.

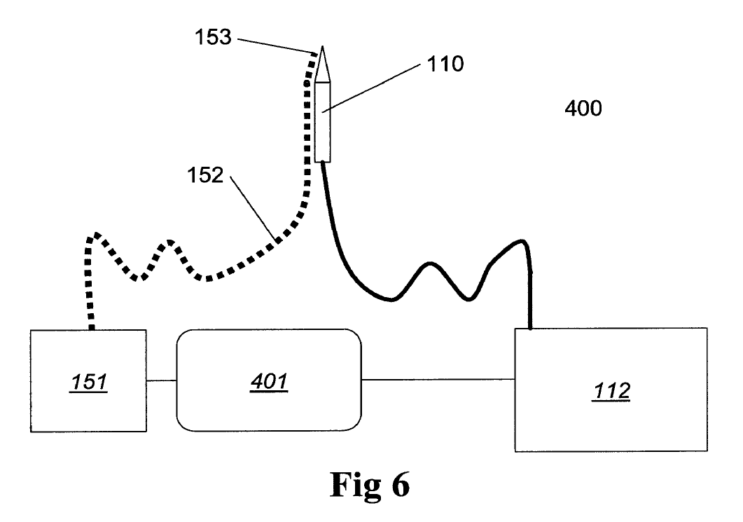

FIG. 6 illustrates a marking system 400 for the intraoperative detection and marking of nerves and other target structures. The detection system 112 and detection probe 110 can be off-the-shelf components utilizing established principles and methods. The marking-system controller 401 reacts to signals detected by the detection system. The marking system controller 401 then activates the dispenser 151 which in turn pumps marking material through the dispenser conduit 152 to the dispensing aperture 153. The dispensing aperture 153 can be adapted and configured to fluidly connect to the pump dispenser 151. Additionally, the dispensing aperture 153 can be associated with one or more sensing elements.

In some instances, the sensing element is a nerve monitoring probe. For example, the sensing element could be a nerve detection unit or a nerve monitoring probe that is commercially available, such as intraoperative neural probes including NIM PRS and NIM spine from Medtronic and the Orthomon from Axon Systems. There are many options clear to those skilled in the art for configuring the marking system controller to respond to signals from a detection system. For example, the audible or light-based notification signal created by the detection system could be detected by an acoustic or optical sensor affixed to the detection system. Alternatively an electrical output of the detection system could be monitored by the marking system controller. The marking system controller can then be adapted and configured to interpret a signal from the nerve monitoring system 112 and send a signal to a marking element to create a mark at the desired location.

Further provided herein is a marking element wherein the marking element is used to create markings associated with a location on the TUT. In FIG. 6, the marking system 400 comprises a marking element comprised of a dispenser 151, dispenser conduit 152 and dispensing aperture 153. Other examples of marking elements include, but are not limited to, ink-deposition systems, ink jet arrays, and electrode arrays for cautery marking, localized electrodeposition, or storage in a database and displayed on a digital display. The marking element may be made using conventional fabrication techniques such as molding or machining, or by micro-fabrication or MEMS.

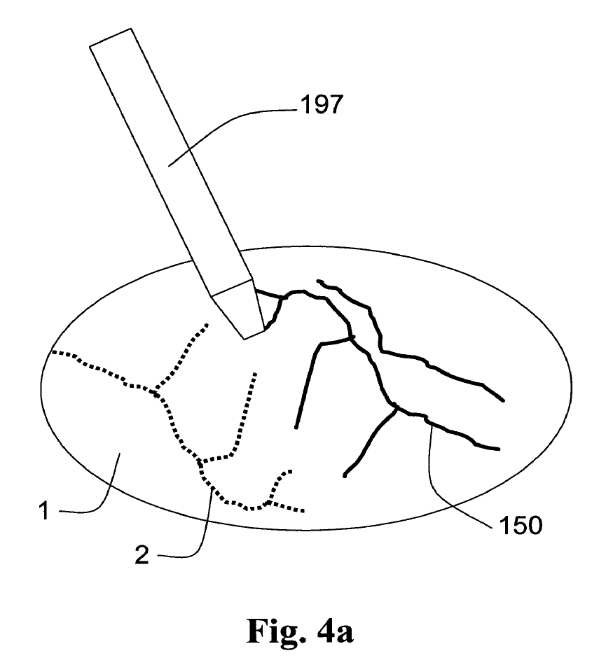

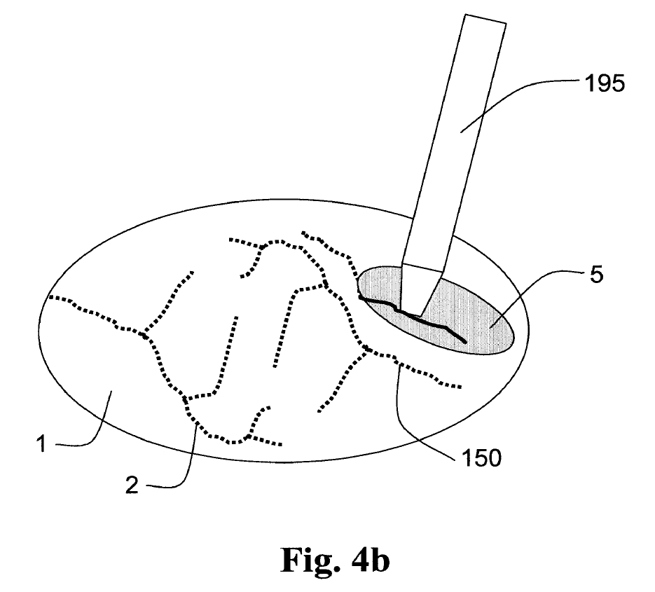

A marking element may also be integrated with a mapping system as shown in FIG. 4A. For example, as illustrated in FIG. 4A a closed-loop marking probe 197 of a mapping system can be used to deposit a marking 150 at the location of a target structure 2 or target structures at the surgical site comprising the area of TUT 1. As described above, the TUT 1 can refers to the portion of the body, or surgical site, that the mapping system or dissection system is being used to analyze. As a further example, in thyroid surgery the TUT is the thyroid gland and adjacent tissue. It is anticipated that the inventions described here may be used multiple times during the course of a single surgical procedure and dissection, so that the TUT may change at various times throughout the procedure as dissection proceeds. Target structures during any surgical procedure include, but are not limited to, elongate organs or other suitable tissue structures located within the TUT, examples of which include a nerve, nerve bundle, vein, artery, ureter, muscles, urethra, or other fascicles, tubes, lymphatic or blood vessels or nodes.

The intraoperative mapping system described herein is able to discriminate between tissue structures and is further able to determine the identification and locations of target structures by transmitting and detecting a signal that physically propagates through the TUT. The properties of the signal can be subsequently used to characterize the structure of the tissue that the signal is passed through. A signal is generated either as a result of a stimulus provided by the excitation element or as a signal intrinsic to the body, e.g., nerve depolarization or signal propagation through the nerve. A signal may include any suitable signal that is capable of being detected and may include, but is not limited to, electrical current, nerve depolarization pulse, thermal conduction, physical vibration, acoustic, electromagnetic or optical transmission.

As shown in FIG. 4A, the marking 150 is associated with a location on the target tissue by a marking element comprising the marking probe 197, and may be used as an indicator for the surgeon of the location of target structures. A marking can also be deposited on a tissue to be removed instead of the target structure. For example, if the target structure is adjacent to a tumor, the tumor can be marked instead of the tissue.

The markings used may be permanent or temporary. Additionally, the markings used may or may not be visible to, for example, a surgeon. The marking made on a tissue may be made with a marking that is placed on, adhered to, or injected into the tissue. As will be appreciated by those skilled in the art, the marking can be a marking that is detectable by the surgeon, by the dissection system and not the surgeon, or by the dissection system and the surgeon. When a marking element is integrated with an intraoperative mapping system, the marking element can, in some instances, deposit a mark that is of one particular type or color. The marking element can also be configured to mark multiple structures. Where multiple structures are marked, more than one color or type of marking may be used to discriminate or distinguish between different tissue regions and/or different tissue structures. Examples of markings that may be used include, but are not limited to, surface cautery, India ink, dyes, or particles. Particles that may be used to mark a tissue further include, but are not limited to, colored particles, light scattering particles, fluorescent particles, quantum dots, carbon nanotubes, paramagnetic particles, ferromagnetic particles, radioactive particles, ferrous particles, metallic particles, bar coded particles, radio frequency identification (RFID) particles, or optically encoded particles. The marking can also include, for example, projected light such as scanned laser images or projected images from a projector or Digital mirror display (DMD), or on a computer monitor or other appropriate display of the TUT where the marking comprises a digital record of marked TUT locations in a data base. Suitable dyes may also be used as a markings and include, but are not limited to, indocyanine green (ICG), methylene blue, 5-aminolevulinic acid (5-ALA), Prussian blue, crystal violet, silver nitrate reduction, direct-current lesions, or radiofrequency lesions. A marking can also have a physical property that inhibits or enhances the action of the dissection element, such as would be achieved by the use of wax.

The dissection element can, for example, be a device that is adapted and configured to dissect, ablate, or remove tissue. As will be appreciated by those skilled in the art, a dissection element can be designed to remove tissue in various ways. For example, a dissection element could use ultrasonic energy, electric current, vibrating blade, thermal ablation, electrochemical reactions, plasma, vacuum, pressure, fluid jets, optical ablation, cryoablation, microwaves, lasers, or any other suitable technique for removing tissue.

Additional marking materials that may be used include dielectrics such as paraffin, wax, sucrose solution, foam, or materials that form a gel upon deposition that can modify the passage of signals such as electric currents, mechanical stresses, ultrasonic energy, or light absorption. Where wax is used, the marking element may be an integrated heated element. The heated element could be used to keep the wax in a liquid form until the wax is deposited at the desired location.

The marking may also selectively bind to certain tissues of interest or target tissues and the marking could be applied to the entire surgical site and then washed away. Wherever the marking selectively binds to the tissue, a pattern or marking may be left on the tissue. Similarly to markings described elsewhere, this would enable visualization by the surgeon or selective dissection. For example, a labeled antibody could be used that selectively binds to a ligand specific to the tissue to be marked.

FIG. 4B illustrates a generalized surgical site where a selective-dissection probe 195 of a selective-dissection system is used to remove tissue 5 while preserving target structures 2 at the TUT 1 site. The purpose of the selective-dissection-system is to enable tissue dissection by the surgeon, while protecting the target structures of the surgical site. This includes one or more controllable dissection element(s) on the selective-dissection probe that dissect, ablate, or remove tissues. Dissection elements can employ different mechanism for removing tissue including, but not limited to, ultrasonic energy, electric current, vibrating blade, moving blade, thermal ablation, electrochemical reactions, vacuum, pressure, cavitation, electrosurgery, fluid jets, optical ablation, cryogenic probe, cryogenic spray, microwaves, or laser.

### III. PROXIMITY SYSTEM

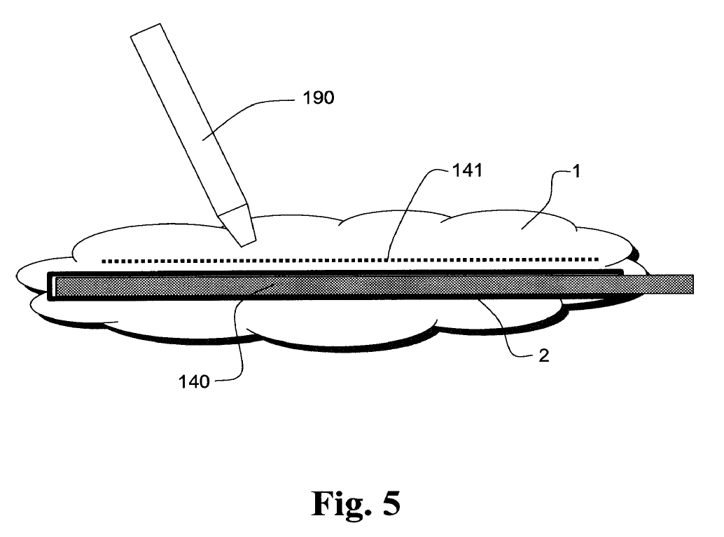

FIG. 5 illustrates a generalized surgical site where a proximity dissection probe 190 is used to dissect the TUT 1 when the proximity dissection probe is outside of the proximity limit 141. The proximity limit can, for example, include a distance between a sensing element and a reference element that triggers a proximity condition. A notification element can be activated by the proximity condition and can include, for example, an element that provides a signal to the surgical staff, another surgical system, or both, that indicates a proximity condition. For example, a sensory notification can be provided to the staff. Examples of indicators of the notification elements include, but are not limited to, an audible tone, a blinking light source such as an LED, or a vibration. As will be appreciated, the proximity condition can occur, for example, when a sensing element is positioned some distance and/or orientation relative to a target tissue, marking element or reference element.

The reference element can be adapted and configured to include a device positioned by a surgeon that is capable of detecting an excitation or stimulus from an excitation element. The reference element can also be generally positioned within or near an anatomical structure and can be left in place during part or all of the surgery. In some instances it may be desirable to leave the reference element in place following the surgery in order to enable a surgeon to locate a specific portion of a surgical site in a subsequent procedure. In some cases it may be desirable to position the reference element or a plurality of reference elements within the body before surgery and potentially use imaging techniques to improve placement accuracy.

For example, the reference could be placed in the urethra during a radical prostatectomy. As will be appreciated by those skilled in the art, the reference element can belong to any of the functional elements described here including, for example, the excitation elements, the sensing elements, the marking elements, and/or dissection elements. The reference element can belong to any of single one of these functional groups or the reference element can belong to multiple functional groups simultaneously. The reference element can be powered either using wires, or alternatively the reference element can be powered through wireless techniques. For example, a radio frequency (RF) or acoustic signal could be used to power the reference element. The probe element could emit an energy source such as RF, and the reference element could respond as a passive radio transponder using, for example, reflected power communications or load modulation. Devices and methods of RFID tags use either low frequency inductively coupled or high frequency interactions and can be powered by the interrogation signal. The spacing between the probe and reference element could be detected by such a system so that it responds at the proximity limit with load modulation or by emitting an RF pulse

As shown herein, the proximity limit 141 is designated as a specified distance from a reference element 140, which in the case of FIG. 5, is inserted into a target structure 2. In an intraoperative proximity system, the reference element 140 is a device positioned by the surgeon within or near either a target structure 2 or any area of tissue and is left in place during either the entire surgery or part of the surgery. The proximity system can be adapted and configured to be a system that detects a spacing or distance between a probe element and a reference element.

A proximity system may, also include a notification element, a marking element, or a dissection element. For example, the reference element 140 could be placed in the urethra during a radical prostatectomy, as shown in FIG. 5. The proximity system can include any of the functional elements described elsewhere including, for example, but not limited to, excitation elements, sensing elements, marking elements, and/or dissection elements. These functional elements can be used either alone or in combination with other functional elements.

The proximity dissection probe 190 includes a probe element and can include a device controlled by a surgeon during the course of a procedure. The probe element can, for example, be an excitation element, a sensing element, a marking element, and/or dissection element. The probe can belong to any one of the previously mentioned functional groups or can belong to multiple functional groups simultaneously. For example, the probe element could belong to both the excitation element and reference element functional groups. For example, the detection element comprises a magnetic recording head, similar to those used on floppy computer disk drives, hard disk drives, or tape drives. The magnetic recording head could further comprise of a coil positioned around a material of high magnetic permeability (e.g. ferrite). If the ferrite material is, for example, shaped like a toroid with a gap in it, time variant magnetic fields at the gap region will be picked up by the coil. Alternatively, the magnetic field sensor can be magnetoresistive or Hall Effect.

A probe element can be paired with a reference element to achieve any combination two functional groups interacting together. For example, a probe element-reference element pair can be adapted and configured to generate any suitable detectable signal and, in some cases, can detect the same suitable detectable signal. Example of signals generated and detected by the probe element-reference element pairs include, but are not limited to, a magnetic coil based pair, where the reference element comprises closed circuit with an oscillating electric current and the probe element comprises a coil, inductor, or magnetic read-head, or a optically based pair, wherein the reference element comprises a light source and the probe element comprises an optical sensor

The reference element of the intraoperative proximity system can either be unpowered, powered using wires connected to an external power supply, or alternatively, through wireless techniques. For example, an RF or an acoustic signal could be used to power the reference element. For example, a probe element on the proximity dissection probe could emit an energy source such as RF waves. The reference element could respond with an RF pulse at a defined frequency after a specified RF intensity threshold is reached. In this case passive electronic circuitry is incorporated within the reference element, similar to un-powered interrogation that has been demonstrated in identification cards and RFID systems. For example, the reference element comprises a closed circuit with an oscillating electric current that generates an electromagnetic field and the proximity dissection probe employs a sensing element composed of a coil, inductor, or magnetic read-head. The characteristics of the reference element can be arranged using established electromagnetic transmission and modulation principles as well as antennae design to improve the sensitivity, accuracy, off axis performance, and resistance to extraneous signals. Frequency coding, modulation, and other techniques known to those in the art can also be used to improve the performance. For example, lock-in-amplification techniques can be used. In an alternative example the reference element comprises a light source such as a light-emitting diode and the sensing element on the proximity dissection probe is an optical sensor. Coding and modulation techniques can also be employed to improve performance, if desired. A reference element or plurality of reference elements may further include, but are not limited to, colored particles, light scattering particles, fluorescent particles, quantum dots, carbon nanotubes, paramagnetic particles, ferromagnetic particles, radioactive particles, ferrous particles, metallic particles, bar coded particles, RFID particles, or optically encoded particles, or liquids gasses or gels with detectable properties

### IV. MAPPING SYSTEM

The purpose of the mapping system is to identify and mark the target structures during surgery, using one or more excitation elements, sensing elements and marking elements to create a map of the tissues located within the TUT. For example, a probe comprising an electrode array combined with an ink-jet print head marks the tissue in response to the impedance.

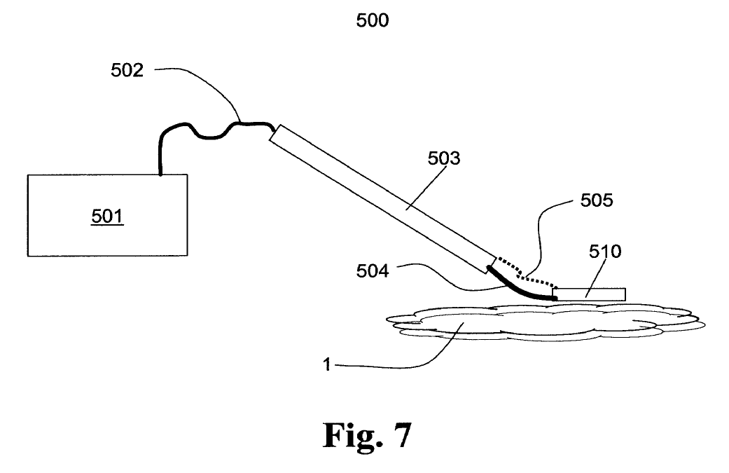

FIG. 7 illustrates a mapping system 500 for the intraoperative detection and marking of nerves and other target structures. The surgeon sweeps the mapping array 510 over the TUT 1 using the probe handle 503 that is affixed to the flexture 504 that provides a flexible connection to said mapping array 510 that employs one or more marking element(s). The distal conduit 505 and proximal conduit 502 provide electrical connection to the mapping-system controller 501.

For example a mapping system could include a simple probe system with one marking element integrated into a single handheld probe with either one sensing element, one excitation element, or both. Other probe-array configurations include configurations comprising: (i) a linear probe array system, in which the sensing-, marking-, and optional excitation elements are arranged in a pattern along one dimension; (ii) a circular probe array system in which the sensing, marking, and optional excitation elements are arranged in a circular pattern. (iii) a brush system wherein the sensing-, marking-, and optional excitation elements are positioned on the ends of the fibers that are attached into a single probe; (iv) sheet array system wherein the scanning-, marking, and optical excitation elements are arranged in a 2-dimensional (2D) pattern on the surface of a flexible sheet substrate; (v) a roller based system; (vi) a MEMS or micro-fabricated system.

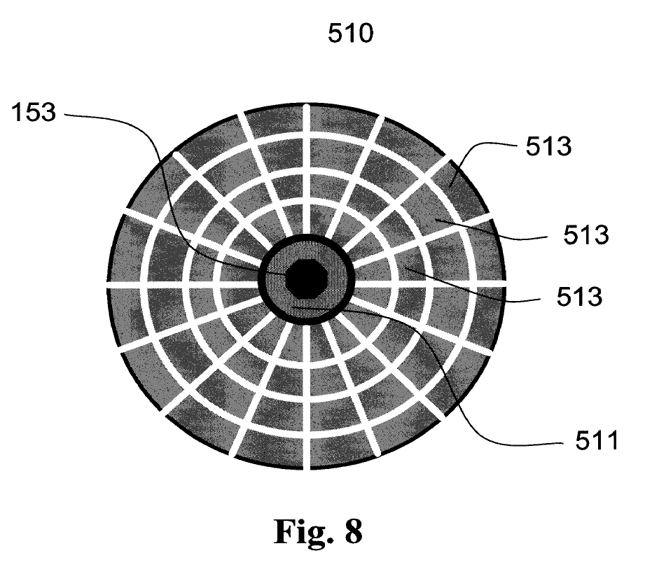

The mapping system can also be adapted to comprise a mapping array 510 as shown in FIG. 8. The mapping array integrates one or more of interrogation means and marking elements. As shown in FIG. 8, a mapping array may, for example, comprise a primary electrode 511, and a plurality of secondary electrodes 513 arranged in a plane around the primary electrode 511. The mapping array may be integrated with a marking system as shown in FIG. 8 where the primary 511 and secondary electrodes 513 are located around the dispensing aperture 153. The primary electrode can, for example, be one or more excitation elements while the secondary electrode can be one or more sensing electrodes. The primary electrode can also be one or more sensing electrodes while the secondary electrodes can be one or more excitation electrodes. Other configurations can also be employed without departing from the scope of the invention. Upon detection of a signal between the primary electrode 511 and the secondary electrodes 513, indicating the presence of a tissue structure, the mapping array can then send a signal to a dispenser, so that a marking can be deposited through the dispensing aperture 153 to the tissue structure identified.

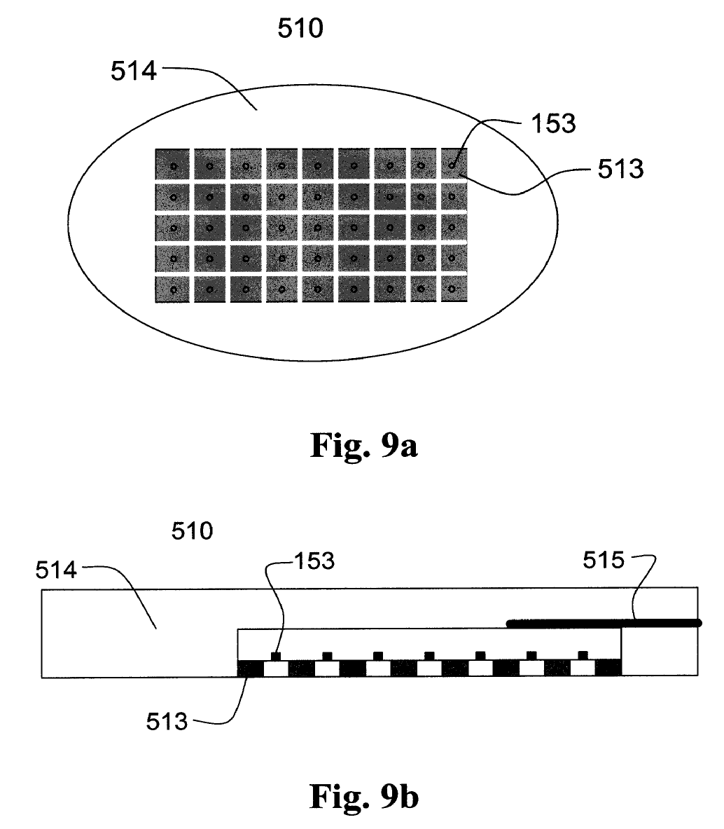

FIG. 9A illustrates another mapping array 510 where a plurality of dispensing apertures 153 are arranged in a mapping array body 514. The dispensing apertures 153 are interposed between a plurality of secondary electrodes 513. FIG. 9B is a cross-sectional illustration of the mapping array 510 shown in FIG. 9A. An electrical interface 515, as shown in FIG. 9B provides communication between the marking system controller and the mapping array 510. The marking elements in communication with the dispensing apertures 153 can, for example, employ ink jet printing technologies. The mapping array 510 in FIGS. 9a and 9b maps multiple points within the TUT area simultaneously by positioning the mapping array body 514 over the TUT.

The mapping system can be adapted to comprise one or more excitation elements, sensing elements, and marking elements. Alternatively, the mapping system can comprise one or more excitation elements, sensing elements, and notification elements. The notification element can include, for example, an element that provides a signal to the surgical staff, another surgical system, or both, that indicates a proximity condition. For example, a sensory notification can be provided to the staff. Examples of indicators of the notification elements include, but are not limited to, an audible tone, a blinking light source such as an LED, or a vibration. As will be appreciated, the proximity condition can occur, for example, when a sensing element is positioned some distance and/or orientation relative to a target tissue, marking element or reference element.

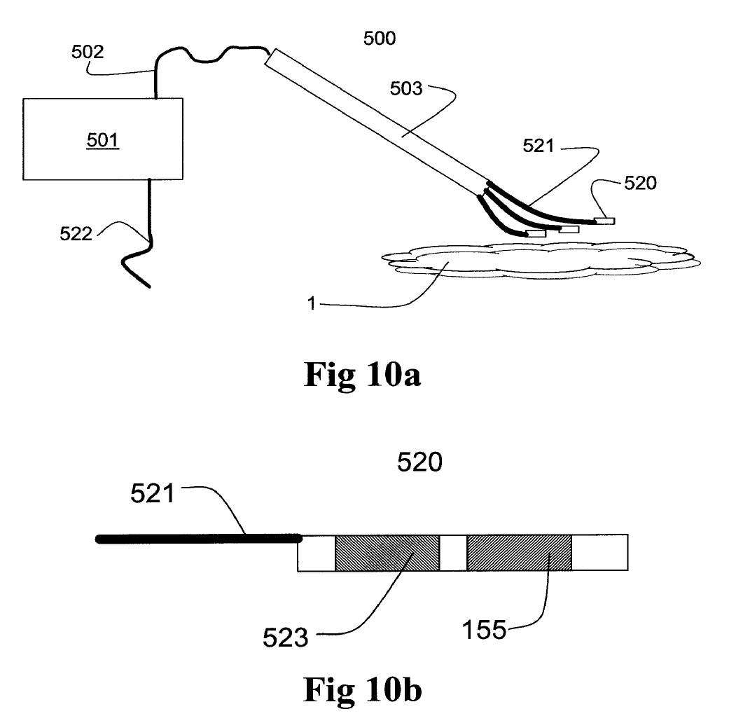

FIG. 10A illustrates a brush-type mapping system 500 for the intraoperative detection and marking of nerves or other target structures. A plurality of mapping elements 520 interrogate and create markings on the TUT 1. A plurality of flexture conduits 521 are affixed between the plurality of mapping elements 520 and the probe handle 503. The mapping system controller 501 is in communication with the probe handle 503 by use with of the proximal system conduit 502. A remote interrogation element 522 is in communication with the mapping system controller 501. The remote interrogation element is positioned on a location of the body remote to the tissue under test and may be a sensor or a stimulator. For example, the remote interrogation element can be an accelerometer, pressure sensor, or electromyographic (EMG) probe used to monitor muscular response to stimulation by the interrogation element 523. Alternatively the remote interrogation element can be a stimulator such as a vibration source used to stimulate a sensory nerve that is monitored using the interrogation element 523. The mapping element 520 is affixed to the distal end of a flexible distal conduit 521 and houses an interrogation element 523 and a marking element 155.

The mapping system herein maps an area of tissue during a surgical procedure by detecting the properties of adjacent tissue as evaluated by analyzing the characteristics of an electrical signal generated between the excitation element and the sensing element. As will be appreciated, the excitation and sensing elements can be configured in a bipolar format. Thus, for example, the electrical impedance of the adjacent tissue area is measured and monitored between the pair of elements. Alternatively, an alternating voltage signal can be provided to an electrode pair while monitoring the passage of the electrical current between the electrodes. A non-depolarizing signal voltage can also be applied to the tissue. A depolarizing voltage signal can also be applied to the adjacent tissue. Where a plurality of excitation elements and sensing elements are used, the excitation elements and sensing elements form pairs. The interrogation of the tissue is then undertaken by measuring and monitoring the impedance between each electrode pair in a sequential manner. The complex electrical impedance can then be measured.

The frequency of the voltage used with the excitation element can be more than about 10 Hz, more than about 30 Hz, more than about 50 Hz, more than about 100 Hz, more than 500 Hz, more than 1000 Hz, more than about 5 kHz, more than about 10 kHz, more than about 50 kHz, more than about 100 kHz, more than about 500 kHz, or more than about 800 kHz. In other cases, the voltage between the excitation element and the sensing element can be less than about 1000 kHz, less than about 700 kHz, less than about 500 kHz, less than about 200 kHz, less than about 100 kHz, less than about 50 kHz, less about 10 kH, less than about 10 kHz, less than about 1 kHz, less than about 500 Hz, less than about 200 Hz, less than about 100 Hz, less than about 50 Hz, or less than about 10 Hz. The frequency of the voltage used with the excitation element can be between about 10 Hz and 1 MHz. In some cases it may be desirable to modulate the voltage between about 10 Hz and 10 kHz.

The mapping system 500 in FIG. 10a, the excitation element, and the sensing element can be configured in a monopolar format. Where a monopolar format is used, the mapping system can comprise a remote interrogation element 522 that serves as an electrical ground with, for example, the remote interrogation element attached to the patient and an excitation element is positioned at the TUT. The electrical impedance of the TUT can then be measured between the excitation element and the remote interrogation element. The excitation electrode is a moveable electrode. A mapping system with a monopolar format may comprise of a plurality of excitation electrodes. Additionally, the plurality of excitation electrodes can be adapted and configured such that the electrodes are moveable.

The mapping system can also be configured to map the TUT by measuring other tissue characteristics besides electrical impedance. The excitation element provides an electrical stimulus to the target tissue. The sensing element can then measure the, e.g., mechanical displacement of the tissue, such as a contraction of muscle fibers in response to the excitation element stimulus. This can be accomplished using a sensing element that is an accelerometer, pressure sensor, stress sensor, or optical sensor.

Tissue properties can also be evaluated wherein the sensing element measures an electrical characteristic of the tissue. The excitation element can utilize magnetic stimulation. The excitation element can also be adapted and configured to utilize acoustic, mechanical, optical, or thermal stimulation and the sensing electrode measures the resulting electrical response in the tissue.

Tissue properties can also be evaluated using detectable properties other than electrical signals. The sensing element can be adapted to detect movement of the tissue, such as repetitive movements or pulsation of arteries and other structures by using optical or mechanical detection of tissue displacement. Detecting movement of the tissue can be done using cantilevers, strain sensors, or image analysis as the sensing element. In some embodiments, the sensing element detects magnetic energy or electromagnetic radiation. The sensing element can be a Hall Effect device. The methods for detecting non-electrical tissue properties can be combined with non-electrical stimulation by the excitation element. The excitation element may use such non-electrical stimulation mechanisms such as thermal stimulation or optical stimulation. The excitation element can use magnetic or electromagnetic stimulation, such as through the use of a solenoid, to stimulate the tissue or items conjugated with the tissue, such as markings of fluorescent particles or paramagnetic beads. The excitation element can use mechanical deformations or vibrations to stimulate the tissue area. Any of these stimulating mechanisms or methods can be used in conjunction with any of the detectable signals mentioned. In addition, any of these excitation and detection mechanisms can be combined with other known forms of detecting tissue characteristics including, but not limited to, ultrasound detection, arrays, or imaging mechanisms. The remote interrogation unit 522 can function as either an excitation element or a sensing element.

Further provided herein is a system for mapping an area of tissue during a surgical procedure comprising: (a) at least one excitation element for interacting with a tissue of interest with a stimulus to generate a detectable signal; (b) at least one sensing element for detecting the presence or absence of a signal; (c) a marking element for creating a detectable mark on the tissue; and (d) a dispenser for dispensing a marking material to the marking element. Also provided herein is a system for mapping an area of tissue during a surgical procedure comprising: (a) at least one interrogation element for stimulating or sensing a detectable signal at a tissue of interest; (b) a remote interrogation element for stimulating or sensing a detectable signal at a tissue of interest; and (c) a marking element for creating a detectable mark associated with a location on a tissue.

### V. DISSECTION SYSTEM

A dissection system for the intraoperative dissection of tissue is also provided. The dissection system has a dissection element that enables tissue dissection and, or ablation with improved targeting of specific tissues while avoiding trauma to other tissues. Existing methods for dissection or ablation include, for example, ultrasonic energy, electric current, vibrating blade, cryoablation, thermal ablation, and laser ablation of tissues.

A selective dissection system (SDS) is provided that includes a system adapted and configured to enable tissue dissection by a surgeon, while protecting target structures. Selective dissection systems may include one or more dissection element(s) and may also include either one or more interrogation elements or one or more marking-detection element(s). Markings may be used that have the property of modifying the action of the dissection element, either inhibiting or enhancing its effect. Moreover, markings with binding properties that select specific tissue types may be used.

The selective dissection system can be combined with any of the intraoperative systems previously described. Such systems and methods include but are not limited to markings that alter the action of the dissection source. For example, a dielectric will block the action of ablation relying on electric current passing through the tissue, and a foam will block the action of ablation relying on acoustic energy Alternatively the selective dissection system can identify target structures by using interrogation elements such as electrical stimulation and accelerometer detection and modulate the dissection element(s) to protect target structures. As will be appreciated, other interrogation-element and dissection-element types as described elsewhere in this disclosure can be used without departing from the scope of the invention.

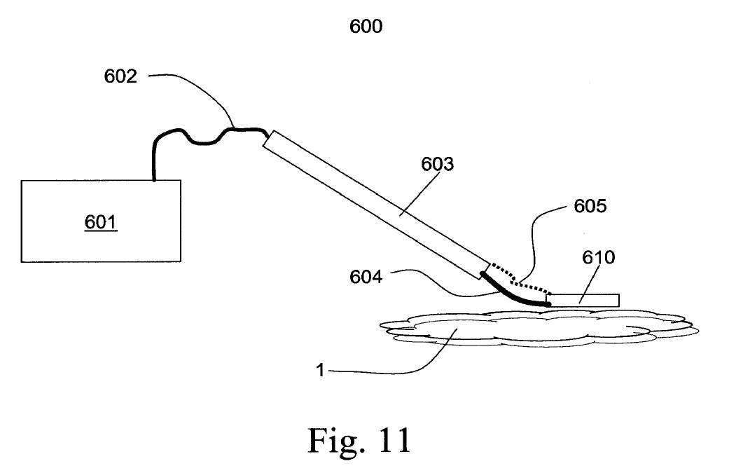

A dissection system 600 is shown in FIG. 11 that includes a selective dissection controller 601 in communication with a proximal system conduit 602 that is in communication with a probe handle 603. A dissection array 610 is applied to the tissue under test 1 and is affixed to the probe handle 603 by the distal conduit 605 and the flexture 604. The dissection array 610 can then selectively dissect and ablate the desired tissue.

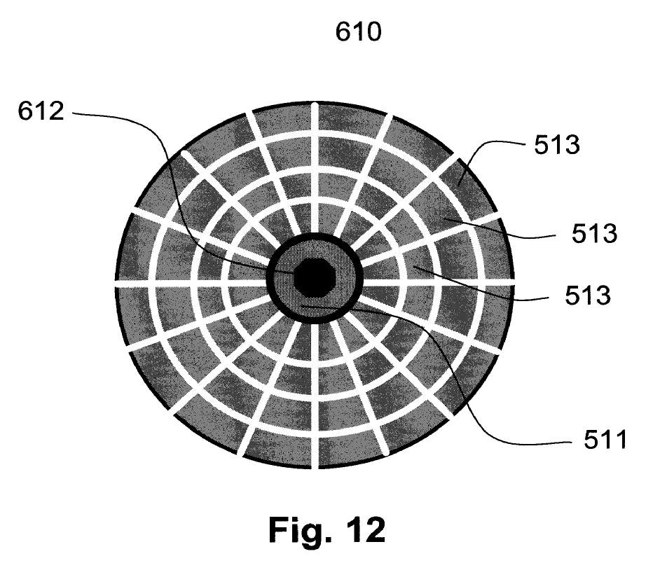

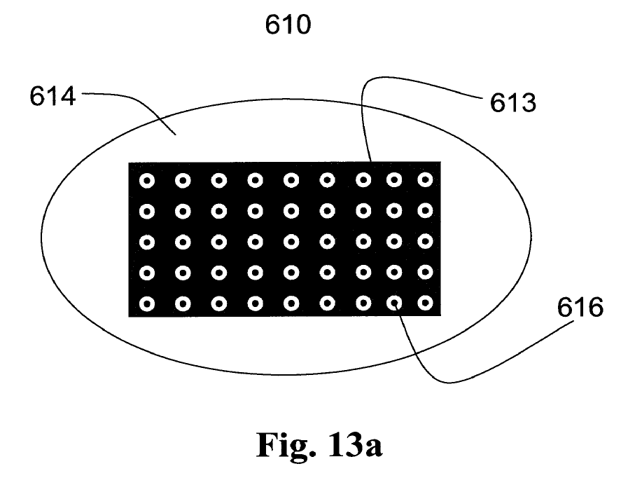

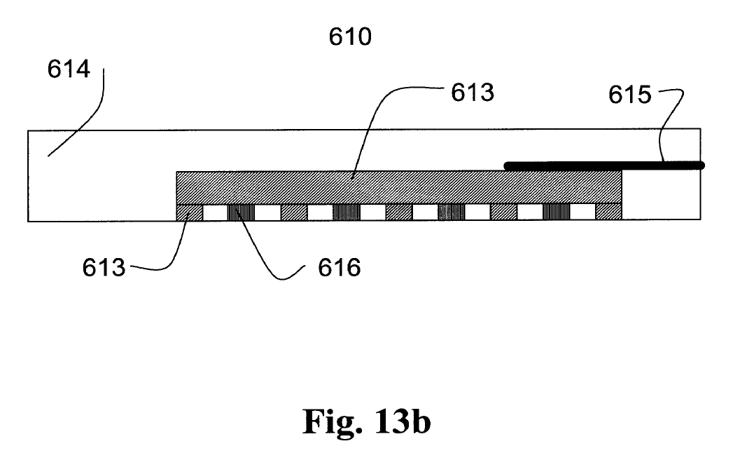

A selective dissection array 610 may be used as shown in FIG. 12. The selective dissection array includes a primary electrode 511 and a plurality of secondary electrodes 513 that are used to identify locations of target tissue as described elsewhere in this disclosure. The dissection element 612 is adjacent to the primary electrode 511. Another selective dissection array FIG. 13A comprises a multiplicity of dissection electrodes 616 disposed on the dissection array body 614 and is further surrounded by a common electrode 613. FIG. 13B illustrates a cross sectional view of the dissection area shown in FIG. 13A. The multiplicity of dissection electrodes 616 are surrounded by the common electrode 613 which is connected to a controller through an electrical interface 615. The electrical signal interface 615 provides the signals for the tissue interrogation and ablation.

In FIG. 12 the interrogation elements are, for example, electrodes made of platinum and the dissection controller unit evaluates the complex electrical impedance between electrodes on opposite sides of the dissection element. A correlation of more than one electrode pair along the same axis results in transient activation of the dissection element. The dissection element is, for example, a monopolar electrode that performs electroablation in conjunction with a ground electrode (not shown) connected between the patient and the controller unit.

The selective dissection system can be combined with other intraoperative systems. A mapping system can also be used to deposit a marker that is an electric insulator. The dissection array comprises multiple dissection elements. An off-the-shelf monopolar electrocautery/ablation system with a small probe is used on tissue that has been marked with electrically insulating marking using a mapping system. The surgeon has enhanced control over tissue ablation as the markings inhibit current to protect tissue selectively.

Provided herein is a system for selectively dissecting tissue comprising: (a) a marking element for creating a detectable mark on a tissue of interest, thereby identifying and marking the location of the tissue; and (b) a dissection element for detecting a marking and selectively dissecting an area of tissue adjacent the marked tissue. The system can further comprise a mapping element. Additionally, the system can be adapted and configured to comprise a proximity system. The system can also be configured so that the tissue that is marked is selectively dissected from the adjacent tissue. Alternatively, the system can also be configured so that the tissue that is marked is selectively further comprise a notification element, and/or an ultrasound, array, or imaging system.

Representative materials and fabrication methods for the devices and systems disclosed herein are summarized in TABLE 1.

TABLE 1
Example Materials and Fabrication Methods for Components

| | | |
|---|---|---|
| Component | Materials | Fabrication Methods |

PROXIMITY SYSTEM

| | | |
|---|---|---|
| Proximity-System Controller | Conventional Digital Electronics | Conventional Digital Electronics |
| Reference Element | A metal coated Foley catheter; made with | Conventional molding and packaging |
| | metalized mylar insert | |
| Probe Element | An inductive proximity sensor | Conventional packaging |
| | MARKING SYSTEM | |
| Marking-System Controller | Conventional Digital Electronics | Conventional Digital Electronics |
| Dispenser | | |
| Fluid Conduit | Polyethylene or polyurethane | Conventional tubing |
| Probe Fixation | clamp made from e.g. polycarbonate, polypropylene | Injection molding |
| Dispensing Aperture | Stainless steel, e.g. 20 gauge needle | Conventional dispensing needle |
| Marking | India Ink in isotonic saline, or others as | |
| | discussed above | |

MAPPING SYSTEM

| | | |
|---|---|---|
| Controller Unit | Conventional Digital and Analog Electronics Output | Conventional Digital Electronics |
| | voltage and current to Proximal Conduit limited | |
| | to levels consistent with human in vivo use | |
| Proximal Conduit | Conventional wire or cable and tubing | conventional |
| | (polyethylene or polyurethane) | |
| Probe Handle | Polyurethane | Injection molding |
| Gimbal | Spring metal, e.g. copper-beryllium or optionally | Conventional (e.g. die cut) |
| | stainless steel (less strain) or polyamide (Kapton) | |
| Distal Conduit | Conventional wire or cable and tubing | Conventional |
| | (polyethylene or polyurethane) | |
| Mapping Array | (components below) | — |
| (Single Marking Channel | | |
| surrounded by Mapping | | |
| Electrodes) | | |
| Substrate | Polyamide (Kapton) sheet (distal conduit | Rolled film with hole die cut in |
| | attached to hole in center) | center |
| Electrodes | Gold | Electroplated and patterned |
| Marking | India Ink in isotonic saline, or others as | |
| | discussed above | |
| Mapping Array | (components below) | |
| (Multiple Marking Channels) | | |
| Mapping-Array Body | Ink Jet Printing array housing | conventional |
| Electrode Array | Gold on polyamide with punched holes for | Rolled polyamide (kapton) sheet, |
| | inkjet nozzles | electroplated, patterned using |
| | | photoresist photomasking/etching, |
| | | and die cut |
| Electrical Interface | Flex-circuit cable (copper in Kapton film) | conventional |
| Printing Array | Conventional inkjet array | |
| Mapping Elements | (components below) | |
| (Brush-like array) | | |
| Controller Unit | Conventional digital and analog electronics; output | Conventional Digital Electronics |
| | voltage and current to proximal conduit limited | |
| | to levels consistent with human in vivo use | |
| Gimbal Conduit | Flex-circuit cable (copper in Kapton film) | Rolled polyamide (kapton) sheet, |
| | | electroplated, patterned using |
| | | photoresist photomasking/etching, |
| | | die cut and bonding using pressure- |
| | | sensitive adhesive |
| Substrate | Extension of gimbal conduit | Above |
| Excitation Element | Electroplated gold electroplated through | electroplating |
| | openings in Kapton top film | |
| Marking Element | Either a pair of contacts electroplated through | Electroplating and photolithography |
| | openings in Kapton top film or patterned gold trace | |

DISSECTION SYSTEM

| | | |
|---|---|---|
| DISSECTION SYSTEM 1 | | |
| Controller Unit | Conventional digital and analog electronics | conventional |
| Proximal Conduit | Wire cable | conventional |
| Probe Handle | Polyurethane | Injection molding |
| Gimbal | Kapton flex circuit | Rolled film, patterned and laminated |
| Distal Conduit | Integrate with gimbal | |
| Dissection Array | | |
| Substrate | Polyimide (Kapton) layers with copper traces, | Rolled film, lamination, die cut, |
| | and holes in overlayer | electroplating |
| Dissection Element | Gold electrodes for electrocautery | Electroplating |
| Interrogation Elements | Gold electrodes | Electroplating |
| DISSECTION SYSTEM 2 | | |
| Dissection Array | | |
| Dissection-Array Body | Polyimide (Kapton) layers with copper traces, | Rolled film, lithographically |
| | and holes in overlayer | patterned copper traces, die cut |
| Electrical Substrate | Integrate with dissection-array body | Rolled film, lithographically |
| | | patterned copper traces, die cut |
| Common Electrode | Electroplated Gold | Rolled film, lithographically |
| | | patterned copper traces, die cut |
| Addressable Electrodes | Electroplated Gold | |
| Electrode Interface | Integrate with dissection-array body | Rolled film, lithographically |
| | | patterned copper traces, die cut |

### VI. KITS

A variety of kits are also contemplated. For example, a kit for marking can be provided. The kit for marking the tissue can comprise, for example, (a) a dispensing aperture for affixing to a nerve monitoring probe; and (b) a biocompatible marking substance; and (c) a marking element for creating a detectable mark associated with a location on the tissue. Furthermore, the kit could include an interface that responds to a signal from the nerve monitoring probe, a set of instructions, and any other component or feature that is desirable or useful to the user.

A kit for mapping an area of tissue during a surgical procedure can comprise, for example, (a) at least one excitation element for interacting with a tissue of interest with a stimulus to generate a detectable signal; (b) at least one sensing element for detecting the presence or absence of a signal; (c) a marking element for creating a detectable mark on the tissue; and (d) a dispenser for dispensing a marking material to the marking element. A kit further comprises a set of instructions. Also provided herein is a kit for mapping an area of tissue during a surgical procedure comprising: (a) at least one excitation element for stimulating a tissue of interest with a stimulus to generate a detectable signal; (b) a remote interrogation element; and (c) a marking element for creating a detectable mark on a tissue. In some embodiments, the kit further comprises a set of instructions.

A kit for selective dissection of tissue can comprise, for example, (a) a marking element for creating a detectable mark on a tissue of interest, thereby identifying and marking the location of the tissue; and (b) a dissection element for detecting a marking and selectively dissecting an area of tissue adjacent the marked tissue. The kit further provides for a mapping element, a proximity system or a notification element.

### VII. METHODS

A variety of methods are also contemplated. One method includes a method for detecting a spacing between a probe element and a target tissue. The method comprises: placing a reference element within a detectable signal range of the target tissue; generating a detectable signal; detecting a signal; and determining a spacing between the probe element and the reference element from a characteristic of the detected signal. The method can further comprise dissecting tissue adjacent the target tissue using a dissection element, for example, where the dissection element is modulated in response to the spacing between the probe element and the reference element. Additionally, the user can be notified of the spacing between the probe element and the reference element. Furthermore, the notification element can be adapted and configured to provide a sensory notification to the user. A marking element can also be activated, if desired, to mark the location of the reference element and target tissue. The reference electrode could be, for example, an electrode placed within the urethra using a catheter. In some cases, a detectable signal is used. The detectable signal can be generated by the probe element, the reference element, or both. Additionally, the probe element, the reference element or both can be adapted and configured to detect the detectable signal. The reference element can also have detectable properties. In some cases, it may be desirable to place the reference element prior to generating a detectable signal. For example, the reference element can be placed in a separate procedure and intraoperative imaging techniques can also be used to assist with this placement.

Another method includes a method for creating a marking on a tissue of interest at a surgical site. For example, the tissue can be marked by: probing a tissue with a sensing element; detecting a signal generated by the tissue being probed; characterizing the tissue being probed to determine if the tissue is a tissue of interest; activating the marking element to mark the tissue if the tissue is of interest; and marking the tissue with a marking element. Suitable tissues of interest include, for example, nerves, nerve bundles, veins, arteries, a ureter, muscles, a urethra, or any other suitable fascicle, tube, lymphatic vessel, node, blood vessel, or any other suitable tissue.

A procedure for creating a mark on a tissue is also included. The procedure can include, for example, stimulating a tissue of interest with a stimulus generated by an excitation element thereby creating a detectable signal; sensing the detectable signal with a sensing element; characterizing the tissue of interest by analyzing the detectable signal; and activating a marking element to mark the tissue of interest if desired, wherein the marking element creates a mark on the tissue for identification and location.

Tissue can also be selectively removed from a surgical site by, for example, positioning a reference element within or adjacent to a tissue of interest; probing the area adjacent the tissue of interest using a probe element wherein a dissection element is operably connected to the probe element; detecting the location of the reference element with the probe element; and dissecting the tissue adjacent the reference element thereby protecting the tissue marked by the reference element.

Alternatively, tissue can be selectively removed by, for example, marking a tissue of interest with a detectable mark using a marking element; detecting the mark using an integrated probe-dissection element wherein the probe component detects the detectable mark; dissecting the tissue adjacent the mark using the integrated probe-dissection element wherein the dissection component dissects the tissue. In yet another example, tissue can be selectively removed by positioning a reference element within or adjacent to a tissue of interest; probing the area adjacent the tissue of interest with a probe element wherein the probe element stimulates the tissue to generate a detectable signal; sensing the detectable signal using the reference element; detecting a spacing between the probe element and the reference element by analyzing a characteristic of the detectable signal; and dissecting the tissue adjacent the reference element with a dissection element wherein the dissection element is capable of being modulated by the spacing between the probe element and the reference element.

### VIII. EXAMPLES

### Example 1

### Sample Tissue

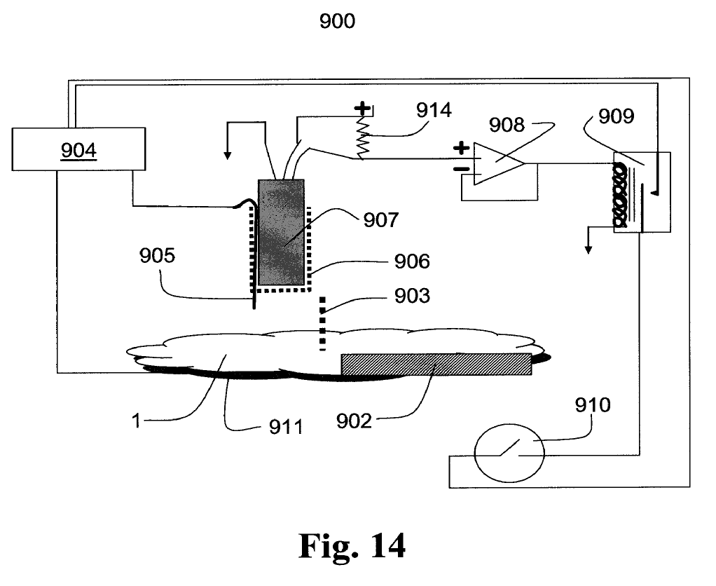

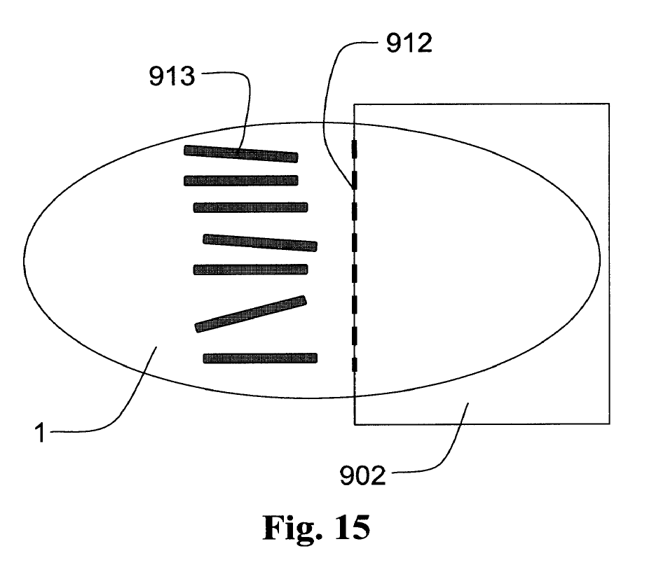

An example proximity system 900 is illustrated in FIG. 14. In this example, the proximity system uses a modified Parkell Sensimatic Electrosurge 500-SE 904 dissection element which was modified so that the activation pedal 910 was in series with a reed relay 909. A Pepper & Fuchs NBN4 inductive proximity sensor element 907 was then affixed to a wire electrode 905 that was connected to the active output of 904 and covered with an insulator 906. Fresh raw chicken breast 1, was used as a test tissue sample and was rested on a grounding electrode 911 and a steel plate reference element 902. The sensing element 907 was expected to be triggered by the reference element 902 at the proximity limit 903. Output of the sensing element 907 was connected to an operational amplifier 908 configured as an inverter and to the +12 V voltage by the 220 k ohm resistor 914. The output of 908 was connected to the relay 909. When the sensor element 907 was not triggered the output of the op amp 908 was −12 V and caused the relay 909 to close. When the sensor element 907 was triggered by proximity to metal the output of the op amp 908 was near 0 V and opened the relay 909. FIG. 15 illustrates a top view of a dissection experiment using the proximity dissection system in FIG. 14. The ink marks 912 on the tissue raw chicken breast 1 indicate the location of the reference element 902. Electroablation was carried out from left to right, resulting in cuts 913 in the tissue 1. As the probe reached the right-most end of the cuts 913, the proximity system automatically disabled the electroablation tool.

### Example 2

### Prostatic Urethra

Another proximity system could be adapted from the proximity system in FIG. 14, using a Foley catheter with, for example, aluminum film coating. The modified catheter would then be inserted into the urethra and would serve as the reference element. This system would be used to preserve the prostatic urethra during a modified radical prostatectomy procedure where the prostate is resected but the urethra is left intact. As noted elsewhere this would have the advantage of minimizing trauma to the urethra and associated structures and is expected to result in shorter recovery times and less urinary incontinence and other complications.

### Example 3

### Radical Prostatectomy

Another proximity system could be used to target a dissection of an interface between the neurovascular bundles 30 and the prostate 10 FIG. 3, during radical prostatectomy. In this example, six neodymium magnets (NdFeB) reference elements coated with Teflon, cylindrically shaped, 500 um in diameter and 1 mm long, would be inserted before radical prostatectomy procedure transanally using intraoperative ultrasound imaging and a biopsy trochar with a tip modified to hold and deploy each reference element. Each reference element would be positioned at the prostate-neurovascular bundle interface 32, three on the left and three on the right. A lap aroscopic tool, such as a tissue retrieval system, would then be adapted to carry, for example, a three axis magnetic sensor such as a Honeywell HMC1023 Three-Axis Magnetic Sensor surface mount in a package with white orientation markings, sensor element. Thereafter, a robotic surgical system, such as the DaVinci robotic surgery system from Intuitive Surgical, can then be used in the surgery. After bladder neck dissection the laparoscopic mounted sensor element is brought into the surgical space and passed manually over the surgical site while the database records the 3-axis magnetic field readings at each point as well as the sensor position from the DaVinci imaging system by virtue of the white orientation markings. An algorithm could then be used to determine the 3-dimensional location of the interface planes 32 and this information would be included as marking on the display during the procedure, helping to better guide the surgeon follow the dissection plane. The reference elements could then be retrieved during the course of the surgery.

### Example 4

### Facial Nerve Identification

In this example, a marking system for use during a retrograde paritodectomy is provided to better identify the facial nerve and its branches. A NIM Response 2.0 Nerve Integrity Monitor (NIM) from Medtronic could be used, as provided for by the manufacturer, where one NIM electrode is placed in the orbicularis oculi muscle to monitor the temporal and zygomatic branches of the facial nerve and a second NIM electrode is placed within the nasolabial groove into the orbicularis oris muscle to monitor the buccal and marginal madibular branches of the facial nerve. The headphone audio output from the NIM system could then be connected to a marking system controller that would then convert the analog output to a digital signal. An IntelliSpense™ Digital Timed Air Dispenser #5100805 would be filled with a solution of India ink and 70% glycerin and the dispensing aperture and attached to the NIM stimulation probe. The marking system controller would then be connected in place of the foot pedal switch to an input which would trigger the dispenser in response an algorithmic interpretation of the NIM system audio output. As the surgeon contacts various regions of the tissue under test, the system would dispense ink at any nerve locations. This system would be used at various stages of the dissection to better aid the surgeon in preserving the facial nerve.

### Example 5

### Cranial Nerve Preservation

A selective dissection system could be configured to assist with cranial nerve preservation during, for example, adenocarcinoma removal, especially when scar tissue is present. A NIM Response 2.0 Nerve Integrity Monitor from Medtronic could be used, as provided for by the manufacturer, in conjunction with two NIM electrode placements to monitor the temporal and zygomatic branches and the buccal and marginal madibular branches of the cranial nerve VII. The headphone audio output from the NIM system could then be connected to a dissection system controller that would generate a dissection interrupt signal when a nerve is detected. A Force FX Electrosurgical Generator from Valleylab could then be used with a bipolar cutting tool that is adapted so that the pencil-based switch connection is connected to the dissection system controller. The bipolar cutting tool is affixed to the NIM stimulator. The surgeon would then dissect the parotid tissue, as is done conventionally, but when the cranial nerve CN VII or it's major branches is stimulated above a threshold set on the dissection system controller, current to the cutting tool would be disabled, thereby protecting the nerve from damage.

### Example 6

### Tumor Margin Identification

An example selective dissection system which could be used in surgical oncology to improve tumor margin is provided. In this example, the patient would be treated with an IV infusion of liposomes 100-200 nM in diameter that contain fluorescent dye marking approximately 5 hrs before surgery. Liposomes of this size range are known to accumulate in tumor growth regions through a process called extravasation. The tumor would then be removed using conventional surgical techniques and the site would be washed with saline. A selective dissection system would be configured with a monopolar loop electrode dissection probe connected to a Force FX Electrosurgical Generator from Valleylab where the electrode is adapted so that the pencil-based switch is connected to the dissection system controller. A pair of optical fibers would be affixed to the dissection probe. The first fiber would be connected to an ultraviolet (UV) excitation source and the second fiber would be connected to an optical sensor that is connected to the dissection controller such that detection of fluorescence near the dissection probe would result in an enabling of the probe current. The dissection probe would be swept over the surgical site where residual tumor is expected and any dyed tissue will be removed or ablated. It is anticipated that this system could be much more sensitive to residual tumor tissue than direct visualization by the surgeon.

### Example 7

### Liver Resection

An example marking system could be used during laparoscopic abdominal surgery during resection of the liver. A marking system controller would be connected to the video image from the laparoscopic camera interrogation element and identify locations of arteries by analyzing the image for regions cyclical tissue movement. A digital overlay marking on the screen would highlight these locations aiding the surgeon in dissecting these vessels and avoiding excess bleeding. It is anticipated that this system would allow the detection of vessels buried below the surface of the tissue.

### Example 8

### Choleocystectomy

An example selective dissection system could be used to avoid bile duct injury during laparoscopic choleocystectomy. A miniature acoustic source would be affixed to an endoscope and positioned in the duodenum near the bile duct entrance. An acoustic sensor would be affixed to a monopolar electrode that is connected to an electrosurgery system. The selective dissection controller will generate pulses at the acoustic source and detect them at the acoustic sensor. When the dissection probe contacts the bile duct the dissection controller will disable the electro ablation current, thereby protecting it from injury.

### IX. REFERENCES

U.S. Pat. No. 4,649,932 for Method and apparatus for deriving currents and potentials representative of the impedances of zones of a body.
U.S. Pat. No. 6,466,817 Nerve proximity and status detection system and method.
U.S. Pat. No. 6,500,128 Nerve movement and status detection system and method.
U.S. Pat. No. 6,564,078 Nerve surveillance cannula systems.
U.S. Pat. No. 6,564,079 Electrode array and skin attachment system for noninvasive nerve location and imaging device.
U.S. Pat. No. 6,609,018 Electrode array and sensor attachment system for noninvasive nerve location and imaging device.
U.S. Pat. No. 6,706,016 Nerve stimulator output control needle with depth determination capability and method of use.
U.S. Pat. No. 7,047,085 Nerve stimulator output control needle with depth determination capability and method of use.
U.S. Pat. No. 7,050,848 Tissue discrimination and applications in medical procedures.
ACS Surgery Principles and Practice 2005, Wiley Souba, et al, editors, WebMD Professional Publishing, New York, 2005.
Braeckman J, et al., “Computer-aided ultrasonography (HistoScanning): a novel technology for locating and characterizing prostate cancer”, BJU Int. 2007 October 8.
Camougis, George, Nerves, Muscles, and Electricity: An Introductory Manual of Electrophysiology, Appleton-Century-Crofts, New York, 1972.
DeFrances C J, Podgornik M N. 2004 National Hospital Discharge Survey. Advance data from vital and health statistics; no 371. Hyattsville, Md.: National Center for Health Statistics. 2006.
Dore B (1995) et al., Urol (Paris). 1995; 101(3):113-21.
Ford et al., “Electrical characteristics of peripheral nerve stimulators implications for nerve localization” Regional Anesthesia (1984) 9:73-77.
Fuller, Joanna K., Surgical Technology Principles and Practice, 4^{th} Edition, Elsevier Saunders, St. Louis, Mo., 2005.
Geddes, L. A., Electrodes and the measurement of bioelectric events, Wiley Interscience, New York, 1972.
Greenblatt et al., “Needle nerve simulator-locator: Nerve blocks with a new instrument for locating nerves” Anesthesia & Analgesia (1962) 41(5):599-602.
Jemal, Ahmedin; Siegel, Rebecca; Ward, Elizabeth; Murray, Taylor; Xu, Jiaquan; and Thun, Michael, “Cancer Statistics, 2007”, CA Cancer J Clin 57:43-66 (2007).
Kantoff P W (2002). Prostate cancer. In DC Dale, DD Federman, eds., Scientific American Medicine, section 12, chap. 9. New York: WebMD.
Lepor, H (2006). “Open versus robotic radical prostatectomy”, Urologic Oncology, 24, 91-93.
Merrick, et al. (2007) Journal of Clinical Oncology, 2007 ASCO Annual Meeting Proceedings Part I. Vol 25, No. 18S (June 20 Supplement), 2007: 15533
Novick, Andrew, et al., Operative Urology and the Cleveland Clinic, Humana Press, Totowa, N.J., (2006).
Plonskey, Robert, Bioelectric Phenomena, McGraw-Hill, New York, 1969, (series: McGrawHill series in bioengineering).
Proteogenex, Inc. (2007) 5839 Green Valley Circle # 103, Culver City, Calif. 90230, http://www.proteogenex.com/.
Ramo, Simon, John Whinner, and Theodore Van Duzer, Fields and Waves in Communication Electronics, John Wiley and Sons, New York, 1965.
Raymond et al., “The nerve seeker: A system for automated nerve localization” Regional Anesthesia (1992) 17(3):151-162.
Smith, J, et al, (2007) “A Comparison of the Incidence and Location of Positive Surgical Margins in Robotic Assisted Laparoscopic Radical Prostatectomy and Open Retropubic Radical Prostatectomy”, J. Urol. Vol. 178, 2385-2390, December 2007.
WebMD2007 http://www.webmd.com/prostate-cancer/radical-prostatectomy
Wells, Maryann P., Surgical Instruments A Pocket Guide, Saunders Elsevier, St. Louis Mo., 2006.
Wilt T (2004). “Prostate cancer (non-metastatic)”, Clinical Evidence (11): 1169-1185.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An intraoperative device for detecting a spacing between a plurality of tools used on a target area of tissue during surgery comprising:
a. a probe element in communication with a signal generator; and
b. a reference element in communication with a signal generator
wherein the reference element is positionable within a detectable signal range.

2. (canceled)

3. The device of claim 1 further comprising a dissection element modulatable in response to a spacing between the probe element and the reference element.

4.-9. (canceled)

10. The device of claim 1 wherein a detectable signal generated by the probe element is selected from a magnetic signal, an optical signal, an acoustic signal, a thermal signal, or any other suitable detectable signal.

11. The device of claim 1 wherein a detectable signal generated by the reference element is selected from a magnetic signal, an optical signal, an acoustic signal, a thermal signal, or any other suitable detectable signal.

12.-13. (canceled)

14. The device of claim 1 wherein the reference element has detectable properties.

15. The device of claim 14 wherein the detectable properties are selected from the group of magnetic, electrical, radioactive, optical, acoustic, or thermal properties.

16.-20. (canceled)

21. The device of claim 1 further comprising a plurality of reference elements.

22.-75. (canceled)

76. An intraoperative device for mapping an area of tissue during a surgical procedure comprising:
a. at least one excitation element for interacting with a tissue of interest with a stimulus to generate a detectable signal;
b. a remote interrogation element; and
c. a marking element for creating a detectable mark on a tissue.

77.-80. (canceled)

81. The device of claim 76 wherein the excitation element stimulus for stimulating the tissue of interest is selected from electrical stimulation, magnetic stimulation, mechanical stimulation, acoustic stimulation, optical stimulation, thermal stimulation, electromagnetic stimulation, mechanical vibration, ultrasound, stimulus arrays, imaging, or any other suitable excitation element stimulus.

82. The device of claim 76 wherein the detectable signal detected from the tissue of interest is selected from an electrical signal, a mechanical signal, an electromagnetic signal, a magnetic signal, a thermal signal, ultrasound, detection arrays, imaging methods, or any other suitable detectable signal.

83.-84. (canceled)

85. The device of claim 76 wherein the detectable mark is selected from a fluorescent dye or radioactive material.

86. The device of claim 76 wherein the detectable mark is selected from a particle, a quantum dot, a carbon nanotube, a paramagnetic particle, a ferromagnetic particle, a metallic particle, a radioactive particle, or a colored particle.

87. The device of claim 76 wherein the detectable mark is selected from paraffin wax, wax, sucrose solution, or any other suitable material that forms a gel upon deposition.

88. The device of claim 76 wherein the detectable mark is stored on a computer.

89.-112. (canceled)

113. A device for selective dissection of tissue comprising:
a. a tissue characterization system for identifying a tissue of interest; and
b. a dissection element for dissecting a tissue adjacent the tissue of interest.

114. The device of claim 113 wherein the tissue characterization system comprises an excitation element and a sensing element.

115.-131. (canceled)
